(19) United States
(12) Patent Application Publication
(10) Pub. No.: US 2025/0302534 A1
(43) Pub. Date: Oct. 2, 2025

(54) IMPLANT ASSISTANCE METHOD AND IMPLANT ASSISTANCE SYSTEM FOR OPTIMISED INSERTION OR JOINT REPLACEMENT

(71) Applicant: Aesculap AG, Tuttlingen (DE)

(72) Inventors: Michael Utz, Tuttlingen (DE); Allan Maas, Konstanz (DE); Thomas Grupp, Denkingen (DE)

(21) Appl. No.: 18/863,062

(22) PCT Filed: May 5, 2023
(86) PCT No.: PCT/EP2023/061979

§ 371 Date: Nov. 5, 2024

(30) Foreign Application Priority Data
May 6, 2022 (DE) 10 2022 111 284.5

(51) Int. Cl. A61B 34/10

(52) U.S. Cl. CPC A61B 34/10; A61B 2034/104; A61B 2034/105; A61B 2034/107; A61B 2034/108

(57) ABSTRACT

A method for simulating and selecting an implant for optimized insertion or joint replacement during a surgical procedure on a patient includes the steps of: reading at least one implant parameter, in particular an implant dimension and/or a weight restriction, of a selected implant model; calculating, based on the at least one implant parameter, simulated loads due to predefined forces and momenta at and on the implant mode; determining, based on the at least one implant parameter, whether load limits of the implant model are adhered to in the simulated loads; and, outputting, via a display device, visual confirmation of a selection of the implant model if the load limits are adhered to, or outputting a visual warning before selection of the implant model if the load limits are exceeded. An implant assistance system and a computer-readable storage medium are used to perform the method.

### CROSS-REFERENCE TO RELATED APPLICATIONS

This application is the United States national stage entry of International Application No. PCT/EP2023/061979, filed on May 5, 2023, and claims priority to German Application No. 10 2022 111 284.5, filed on May 6, 2022. The contents of International Application No. PCT/EP2023/061979 and German Application No. 10 2022 111 284.5 are incorporated by reference herein in their entireties.

### FIELD

The present disclosure relates to an implant assistance method for optimized insertion of an implant or (partial or complete) joint replacement via an implant during a surgical intervention in a patient. In addition, the disclosure relates to an implant assistance system as well as a computer-readable storage medium.

### BACKGROUND

Preoperative planning of interventions, which may need to be (re-)adjusted intraoperatively, is currently usually based solely on the anatomical conditions or anatomy of the patient on the one hand and implant dimensions on the other. The patient's weight restrictions defined by the manufacturer for special implant sizes must be observed. However, specifying a weight restriction as the sole parameter for selecting the implant is usually too one-sided and too imprecise and can lead to the implant not being selected that is (optimally) suitable for the patient. As a result, the selected implant does not meet the patient's requirements, in particular mechanical requirements, which can lead to serious complications later on. The correct selection of an implant and a prediction of a possible complication is therefore of crucial importance for successful implantation of the implant, which should fulfill its function effectively and safely over the entire lifetime of the patient.

### SUMMARY

It is therefore the objects and objectives of the present disclosure to provide an implant assistance method and implant assistance system as well as a computer-readable storage medium for optimized joint replacement, which provides an even better selection of an implant based on parameters that is optimally adapted to the patient. Another partial object is to check the implant in advance to determine whether it is suitable for the patient and the application.

The objects of the present disclosure are solved with respect to a generic implant assistance method, with respect to a generic implant assistance system, and with respect to a computer-readable storage medium.

In contrast to the prior art, in which mechanical load limits of the implants are regularly not taken into account, in particular depending on a respective installation situation of the implant, a basic idea of the present disclosure thus provides that such load limits are taken into account accordingly for the selection of an implant. Attention is paid to the dynamic forces and momenta acting on the implant over the course of the load profiles (simulated loads), for example in a load profile of a patient walking or running, in particular depending on the patient's weight and the installation situation (i.e. the position and orientation of the implant in the patient's body). These parameters of the load profiles with the (dynamic) forces and momenta in a load profile are of decisive importance for successful implantation. In the simplest case, only static forces and momenta on the implant may be taken into account.

The present disclosure thus proposes to perform a computer-aided simulation of the loads with the forces and momenta at or on the selected implant or implant model as part of the preoperative or intraoperative planning. The load limits of the implant component(s) and, in particular, patient-specific parameters are taken into account. In this way, the best treatment for the patient can be achieved without exceeding the load limits of the implant or the implant components of the implant (e.g. the implant components for a knee replacement).

In particular, the pre- and intraoperative planning of a TKA (total knee arthroplasty) operation is enhanced by simulating the forces and momenta on the implant.

In still other words, a computer-implemented implant assistance method and an assistance system for a simulation and selection of at least one implant for optimized insertion or joint replacement in a surgical intervention in a patient is proposed, comprising the steps or respectively a correspondingly adapted control unit: reading in at least one implant parameter, in particular at least one implant dimension and/or a weight restriction, of a selected implant model; calculating, by a calculation unit and on the basis of the at least one read-in implant parameter, of simulated loads by predefined forces and momenta on and at the implant model; determining, by the calculation unit and on the basis of the at least one read-in implant parameter, whether load limits of the implant model are complied with for the simulated loads; and outputting, by a display device, for example a surgical monitor, of a visual confirmation of a selection of the implant model when the load limits are complied with or outputting a visual warning not to use the implant model when the load limits are exceeded. In particular, an acoustic warning signal/alarm may be emitted via a loudspeaker in addition to the visual warning.

The term ‘implant model’ is understood here to mean a computer-readable, digital model of the implant, which simulates and attempts to approximate the real implant of the real world in the digital world. In particular, the implant model is available as a three-dimensional CAD model, which is used to perform the numerical calculations with regard to mechanical loading.

Advantageous embodiments are explained below.

According to a preferred embodiment, the step of calculating of simulated loads may reproduce/simulate a load situation of a dynamic activity, in particular walking or running, on the implant model, in particular a load situation scaled to 100 kg, which was preferably obtained from measurement data of an instrumented knee prosthesis. In other words, in one embodiment, the simulation may reproduce the average load situation scaled to 100 kg during at least one dynamic activity, which was obtained from measurement data of instrumented knee prostheses. In this way, a subsequent normal movement of the patient can be simulated and the forces and momenta on the implant caused by the movement can be simulated (as simulated loads). The assistance method and the assistance system can thus be used to check whether the selected implant and, further preferably, an implant alignment is suitable for these dynamic temporal load profiles with the forces and momenta.

According to a further preferred embodiment, in the step of calculating of simulated loads, a standardized load test according to standard ISO 14879-1 or according to standard ISO 14243 can be used as a basis and is simulated and calculated accordingly. Preferably, tests according to at least one standard ISO 14879-1 (Implants for surgery—Total knee-joint prostheses) or ISO 14243 (Implants for surgery—Wear of total knee-joint prostheses) can be mapped and calculated in the simulated loads. The implant components are simulated. In this way, a standardized simulated load is provided in accordance with a standard in order to obtain a standardized, meaningful and reliable calculation. These simulated loads of the standards cover a common standard range.

Preferably, the at least one implant parameter may be an implant type, and/or an implant size, and/or an implant dimension, and/or a position and orientation (pose) of the at least one implant. In other words, in particular an implant type, an implant size, a position and/or an orientation of the individual (implant) components of the selected implant can be taken from the preoperative planning.

In particular, the implant assistance method may evaluate a plurality of implants or implant components and read in a plurality of implant parameters for different implants or implant components.

Preferably, the patient's weight and the installation situation are read in as parameters and are used as the basis for the calculation. The simulated loads are therefore also adjusted in particular on the basis of the patient's actual weight and at least one muscular parameter. The simulation is therefore calculated individually for each patient. One embodiment shows as a result whether the forces and momenta determined are within the range of values previously defined as permissible for the implant. If the values are higher, the target service life of the implant is unlikely to be achieved, which is to be avoided. This means that the loads on the implant system (e.g. anchoring stability) and the expected implant service life (e.g. increased wear) are also included in the preoperative planning for the first time. In particular, the simulated load with the forces and momenta on the implant model can correlate with the patient's weight, i.e. when a patient's weight is higher, correspondingly higher forces and momenta on the implant model are taken as a basis.

According to one embodiment, the step of calculating of simulated loads may include the (actual) weight of the patient and/or a muscular parameter of the patient to provide a patient-specific calculation and selection.

According to a further embodiment, the method may further comprise the step of reading in a patient anatomy by reading in three-dimensional images/picture data of the patient, in particular MRI images and/or CT images and/or X-ray images, to create a simulation model with a patient model and the implant model, and performing the step of calculating more accurately, in particular based on a three-dimensional portion of a skeletal model and/or a musculoskeletal model of the patient. In other words, preferably it is provided to integrate an individual patient anatomy using images/picture data. In this case, the simulation model with the implant model is extended by a patient model, in particular by bony elements for the femur and tibia. This extended simulation model with the implant model on the one hand and the patient model on the other hand now allows an even more precise and individually adapted calculation of the simulated loads on the implant model using numerical calculations. The geometry of the bones in the simulation can preferably be adapted to the patient anatomy on the basis of X-ray images and/or CT data and/or MRI data. This process can preferably be simplified and improved by using an SSM (Statistical Shape Model), especially when using X-ray images. Here, a statistical 3D model is deformed based on the dimensions from the 2D X-ray image so that it replicates the patient's bone as closely as possible in 3D. In other words, the dimensional parameters of a three-dimensional ‘sample model’ are adjusted on the basis of a two-dimensional X-ray image and approximated accordingly.

According to a preferred embodiment, the simulation, i.e. the step calculating the simulated loads, may be carried out intraoperatively. This makes it possible to integrate intraoperative conditions and data, which can only be collected intraoperatively, into the simulation. In particular, a three-dimensional bone geometry can be calculated using palpated landmarks on the patient, which are detected in space by the navigation system, preferably using a statistical shape model (SSM), and then used or taken as a basis in the simulation.

In particular, an intraoperative ligament situation and/or a measured joint gap as a function of applied forces and flexion angles may also be taken into account in the simulation or calculation of the simulated loads.

Preferably, in the step of outputting the visual warning when the load limits are exceeded, the assistance method may further comprise the step of: outputting, on the basis of a stored implant data set, of an implant model (suggestion) with a modified implant size and/or implant orientation that complies with the load limits. For example, with a selected implant size of 5, the next larger and suitable implant size of 4 can be suggested and visually output accordingly. In other words, if the loads are too high or the load limits are exceeded, the simulation result may also contain suggestions as to how these can be reduced. This can be achieved, for example, by changing the implant size or implant orientation. A visual output may preferably also be provided on a section of the display device, in particular a navigation screen. If, for example, the implant parameters and/or the parameters for positioning and aligning of the implant are changed during intraoperative navigation, a corresponding output can be used to indicate whether the selected parameters or values are still within the load limits or not. In particular, this is advantageous if a corresponding simulation becomes part of the navigation application and supports the surgeon accordingly. Preferably, the step of outputting a suggestion of an implant model may include the steps of outputting, on a sub-area of the display device, in particular the navigation screen, of an implant model and a colored (volume) area or corridor for the implant model, with different degrees of compatibility/fit assessments. In particular, the navigation screen may display a red corridor in the navigation data for the area that was determined by the calculation to be unsuitable for the implant model, a yellow corridor can be displayed for an area of the implant model that was calculated to be average for the implant, and a green corridor can be calculated for a (volume) area of the implant model that was determined to be good for the implant model. In this way, a surgeon is given a choice of options for positioning and aligning the implant model in advance. Preferably, an implant size may also be changed and recalculated accordingly and displayed visually in the navigation data. In particular, such a corridor may be configured in the form of a virtual volume that is partially transparent in order to display the surrounding tissue accordingly. Preferably, the transparency is less than 50%. By coloring the corridor accordingly in red, yellow and/or green (in one embodiment, the surgeon (user) can toggle through the three corridors red, yellow and green in order to display the implant model individually in relation to the corridors), a simple and intuitive representation of possible positioning and orientation can be provided to the surgeon in order to make planning and execution even more efficient and to further minimize intervention time. This may be done both preoperatively and interoperatively. In particular, calculated volume models may be used intraoperatively to display this calculated, particularly suitable corridor for an implant model exactly in real time with the surrounding tissue or bone. The decision regarding the implant and the parameters is ultimately made by the surgeon, but is supported very well in advance.

According to one embodiment, the method may further comprise the steps of detecting, in particular before an incision (intra-operatively) or pre-operatively, kinematics of the joint of the patient by a navigation system or a tracking system, in particular via markers attached to the joint or inertial measurement units (IMU) attached to the joint; determining, based on the detected kinematics of the patient, a kinematic phenotype; and outputting, based on the phenotype, of a suggestion of an implant type and/or an implant alignment. In this embodiment, the patient kinematics of the relevant area are recorded in particular intraoperatively, but before the joint incision, using a navigation system. In particular, markers are attached to the patient's femur and tibia via a stab incision. Alternatively, the kinematics may also be detected using IMUs attached to the joint. Standard clinical tests, in particular the Lachmann test, are then preferably performed and detected. After attaching the markers, the leg is passively moved from extension to flexion and back several times in one variant. Maximum varus stress, maximum valgus stress or respectively no lateral load are applied to the joint. These maximum values with regard to varus and valgus as well as the natural varus and valgus, without lateral force application, over the entire possible flexion allow a characterization of the soft tissue situation and can then be taken into account in the simulation. In this way, the simulation model is mapped even more accurately with the implant model and an even better statement can be made on the selection of the implant model. Furthermore, one embodiment may include the preoperative detection of a patient's kinematics, in particular their kinematic phenotype. If the patient's movement data is detected preoperatively, the kinematic phenotype can be determined from this. According to the literature, there are various phenotypes into which kinematic patterns can be categorized. The different phenotypes are taken into account when selecting the implant type and sometimes require different implant alignments. Only in this way can the patient's preoperative, natural kinematics be successfully reproduced postoperatively. For this reason, the patient's kinematic data can also be used in the simulation. The aim of the simulation and calculation of the simulated loads is then to comply with the load limits of the implant over the entire activity profile, subject to the constraint of reconstructing the patient's natural or optimal kinematics.

According to a further embodiment, in addition to the at least one implant parameter, a further parameter for ground reaction forces and/or electromyography data (EMG data) and/or moving images, in particular of a fluoroscope, can be read in in order to simulate a course of patient kinematics and to obtain a time course of the predefined forces and momenta on the implant by inverse kinematics, which are used as basis as the simulated loads in the step of calculating. In the embodiments described above, the simulation is carried out in particular with only skeletal models, i.e. models that do not have any muscle forces, tendon attachments and the like. In this embodiment, however, ground reaction forces, EMG data and/or moving images, e.g. from a fluoroscope, are used in addition to the (basic) parameters. The data added in this way creates a comprehensive picture of patient kinematics over the entire (temporal) course of the recorded activity. Using the inverse kinematics approach, the forces and momenta in the joint or on the implant can now be calculated in the simulation and analyzed over time. This can be done particularly accurately if the ligament attachments from the 3D-picture data used to create the 3D bone model are also determined beforehand and used in a musculoskeletal model.

Preferably, the assistance method may further comprise the steps:
creating a multidimensional matrix based on a variance of input parameters; determining limits/boundary parameters that are to be complied with in the respective combination of input parameters so that the load limits are complied with; preferably outputting, as a function of a patient's weight and an implant size, of an output parameter varus/valgus angle at which the implant can be inserted without exceeding the load limits; and preferably outputting a confirmation if a combination of the input parameters is within the defined limits or outputting a warning if a combination of the input parameters is outside the defined limits. In order to keep the simulation and calculation time as short as possible, especially intraoperatively, all the necessary calculations can be made in advance. A multidimensional matrix can be created based on the variances of the input parameters. In this matrix, the boundary parameters are now determined for each input variable, which must be adhered to in the respective combination of input variables in order to ensure that the loads remain below the previously defined limits. Depending on the patient's weight, this results in a maximum varus-valgus angle for each implant size in particular, under which the prosthesis can be fitted without exceeding the load limits. If all these limits are determined for all input parameters, there is no need for intraoperative simulation and calculation. It is only necessary to check whether the respective parameter combination is within or outside the defined limits. The complexity of this matrix increases with each input parameter, as the dimensions of the matrix correspond to the number of input parameters. Accordingly, there is a degree of complexity at which it is easier to calculate the simulation directly with the corresponding input parameters using a simulation model, such as a numerical simulation of a CAD model.

Preferably, the method may comprise a step of creating a multidimensional matrix comprising: creating an M×N matrix, where M is the number of input parameters, and N is at least three with an entry variance input parameter, an entry lower limit and an entry upper limit, where the entered values of the lower limit and/or the upper limit as well as the variances are in particular static values which have already been determined in advance for the specific combination of input parameters in order to enable a fast calculation.

In particular, the implant is a knee joint implant or a shoulder implant or a hip implant or a spinal implant or an ankle joint implant. Preferably, the implant may also be a vascular implant. The approach of simulating and calculating the loads on the implant or implant model during preoperative planning and taking load limits into account accordingly can be applied in particular to joint implants such as a shoulder implant, hip implant, spinal implant and ankle implant. If sufficient data is available and/or detected, the design of a vascular implant can also be simulated. This is where the detection and simulation of volume flows, pressures and viscosity becomes relevant. Both the dimensions and the design of the implant can then be simulated.

The present disclosure thus deals in particular with a patient-specific simulation of implant-loads using kinematic phenotypes and a gait analysis, in particular a ‘Smart Gait Lab’ gait analysis.

The objects of the present disclosure are solved with respect to an implant assistance system for a simulation and selection of at least one implant for optimized insertion or joint replacement in a surgical intervention in a patient, comprising a display device, in particular a surgical monitor, for outputting a visual content, in that it comprises: a control unit adapted for reading in at least one implant parameter, in particular at least one implant dimension and/or a weight restriction, of a selected implant model; calculating, on the basis of the at least one read-in implant parameter, of simulated loads by predefined forces and momenta on and at the implant model; determining, on the basis of the at least one read-in implant parameter, whether load limits of the implant model are complied with in the simulated loads; and outputting, by the display device, of a visual confirmation of a selection of the implant model when the load limits are complied with or outputting a visual warning not to use the implant model when the load limits are exceeded.

With respect to a computer-readable storage medium, the objects of the present disclosure are solved by comprising instructions which, when executed by a computer, cause the computer to execute the assistance method according to the present disclosure.

Any disclosure related to the assistance method of the present disclosure also applies to the assistance system of the present disclosure, as well as vice versa.

### BRIEF DESCRIPTION OF THE DRAWINGS

The present disclosure is explained in more detail below with reference to preferred embodiments with the aid of Figures, of which:

FIG. 1 shows a schematic view of an implant assistance system according to a preferred embodiment of the present disclosure; and

FIG. 2 shows a flow diagram of an implant assistance method according to a preferred embodiment of the present disclosure.

The Figures are schematic in nature and are only intended to aid understanding of the invention. Identical elements are marked with the same reference signs. The features of the various embodiments can be interchanged.

### DETAILED DESCRIPTION

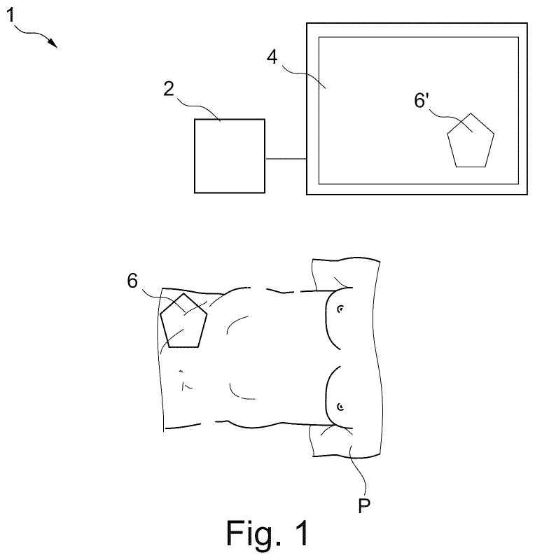

FIG. 1 shows a schematic view of an implant assistance system 1 (hereinafter referred to as assistance system) according to a preferred embodiment. The implant assistance system 1 of the present embodiment is adapted for a simulation and selection of at least one implant 6 for optimized insertion or joint replacement during a surgical intervention in a patient P. Specifically, it comprises a display device 4 in the form of a surgical monitor in order to output a visual content for a medical professional, in particular a surgeon.

Furthermore, the assistance system 1 has a control unit 2 which is specially adapted to read in at least one implant parameter in the form of an implant dimension and a weight restriction of a selected implant model 6′. This implant model 6′ is a digital counterpart to a real implant and is intended to reproduce this implant 6, which is inserted into the patient intraoperatively, in a computer-readable manner. In the present embodiment, the implant 6 is a hip joint and the implant model 6′ is a corresponding digital hip implant model.

The control unit 2 has a calculation unit as a sub-unit (not shown), which calculates simulated loads based on the read-in implant parameters using predefined forces and momenta on and at the implant model 6′ and performs numerical simulations with regard to mechanical loads. On the basis of the at least one read-in implant parameter, the control unit 2 (or the calculation unit) determines whether the load limits associated with the implant model 6′ are complied with for the numerically simulated loads.

Finally, the control unit 2 controls the display device 4 to output the result of the simulation and calculation to the healthcare professional.

If the control unit 2 determines that the load limits for the simulated loads on the implant model 6′ are complied with, it issues a visual confirmation via the OR monitor, for example in the form of a text or a graphic, which the surgeon can use to clearly determine the admissibility of the selected implant model 6′.

However, if control unit 2 determines that the load limits have been exceeded, it controls the OP monitor in such a way that a visual warning is issued before the implant model is selected. In addition, an acoustic warning signal is emitted via a loudspeaker (not shown).

In this way, a load, in particular a predefined load profile, is already taken into account when selecting the implant or implant model and feedback is given to the healthcare professional as to whether the selected implant 6 or implant model 6′ is suitable for the patient. In this way, an optimal selection with a corresponding service life of the implant 6 can be individually assessed in advance and the safety of the patient P can be increased.

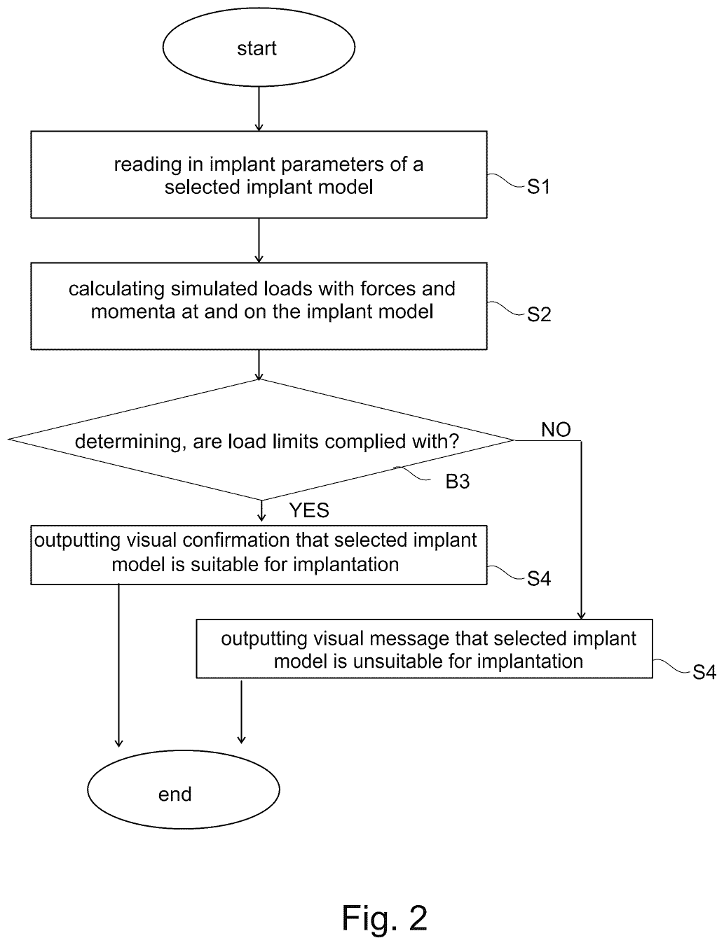

FIG. 2 shows a flowchart of a preferred embodiment of a computer-implemented implant assistance method (hereinafter only referred to as method) for a simulation and selection of at least one implant 6 for optimized insertion or joint replacement in a surgical intervention in a patient P.

In a first step S1, the method performs reading in of at least one implant parameter, in this case an implant dimension and a weight restriction, of a selected implant model 6′.

In a step S2, simulated loads are calculated by a calculation unit (or a control unit 2 with calculation unit) and, on the basis of the at least one read-in implant parameter, by predefined forces and momenta on and at the implant model 6′.

In a condition B3, it is determined in the method, by the calculation unit and on the basis of the at least one read-in implant parameter, whether load limits of the implant model 6′ are complied with for the simulated loads.

If condition B3 is positive and the load limits are complied with, a visual confirmation of a selection of the implant model 6′ is output via a display device 4 in step S4.

If, on the other hand, it is determined in condition B3 that at least one load limit has been exceeded, the method proceeds to step S5, in which a visual warning is output via the display device before the implant model 6′ is selected.

In particular, the specific exceeding of the force or momentum at the specific location of the implant model 6′ may also be output in step S5 in order to provide the healthcare professional with feedback for further optimization of a selection of the implant model. The control unit 2 may also be adapted to provide a suggestion of a further second implant model that complies with the load limits based on preoperative data and the selection of the implant model. Alternatively or additionally, control unit 2 may be adapted to calculate an implant orientation and output an optimal implant orientation that best complies with the load limits.

### LIST OF REFERENCE SIGNS

1 implant assistance system
2 control unit
4 display device
6 implant
6′ implant model
P patient
S1 step of reading in implant parameter
S2 calculation simulated loads on and on implant model
B3 determining, are load limits complied with?
S4 outputting a confirmation of the selection of the implant model
S5 outputting a warning before selecting the implant model

1.-12. (canceled)

13. A computer-implemented implant assistance method for a simulation and selection of at least one implant for optimized insertion or joint replacement in a surgical intervention in a patient, the computer-implemented implant assistance method comprising the steps of:
reading in of at least one implant parameter of an implant model, wherein the at least one implant parameter comprises an implant type;
calculating simulated dynamic loads by a calculation unit and based on the at least one implant parameter by predefined forces and momenta on and at the implant model of a load situation of a dynamic activity;
determining, by the calculation unit and based on the at least one implant parameter, whether load limits of the implant model are complied with for the simulated dynamic loads; and
outputting, by a display device, of a visual confirmation of a selection of the implant model when the load limits are complied with or outputting a visual warning before the selection of the implant model when the load limits are exceeded.

14. The computer-implemented implant assistance method according to claim 13, wherein the step of calculating simulated dynamic loads of a load situation of a dynamic activity that is reproducing a load situation scaled to 100 kg on the implant model, which is obtained from measurement data of an instrumented knee prosthesis.

15. The computer-implemented implant assistance method according to claim 13, wherein, in the step of calculating simulated dynamic loads, a standardized load test according to standard ISO 14879-1 or according to standard ISO 14243 is used as a basis and is simulated and calculated accordingly.

16. The computer-implemented implant assistance method according to claim 13, wherein the at least one implant parameter comprises an implant size and/or an implant dimension of the implant model.

17. The computer-implemented implant assistance method according to claim 16, wherein the at least one implant parameter further comprises a position and orientation of the implant model.

18. The computer-implemented implant assistance method according to claim 17, wherein the at least one implant parameter further comprises a weight restriction of the implant model.

19. The computer-implemented implant assistance method according to claim 13, wherein the step of calculating simulated dynamic loads includes a weight of the patient and/or a muscular parameter of the patient as patient parameters and scales the simulated dynamic loads with the weight to provide a patient-specific calculation and selection.

20. The computer-implemented implant assistance method according to claim 13, further comprising the steps of:
reading in a patient anatomy by reading in three-dimensional images of the patient of MRI images and/or CT images and/or X-ray images to create a simulation model with a patient model and the implant model; and
performing the step of calculating simulated dynamic loads more accurately, based on a three-dimensional portion of a skeletal model and/or a musculoskeletal model of the patient.

21. The computer-implemented implant assistance method according to claim 13, further comprising the step of outputting the visual warning when the load limits are exceeded:
outputting, based on a stored implant-data set, an implant model with a modified implant size and/or implant orientation that complies with the load limits.

22. The computer-implemented implant assistance method according to claim 13, further comprising the steps of:
detecting, before an incision, kinematics of a joint of the patient by a navigation system or a tracking system via detectable markers attached to the joint or via inertial measuring units attached to the joint;
determining a kinematic phenotype based on the kinematics of the joint of the patient; and
outputting a suggestion of implant type and/or implant alignment based on the kinematic phenotype.

23. The computer-implemented implant assistance method according to claim 13, wherein, in addition to the at least one implant parameter, a further parameter for ground reaction forces and/or electromyography data and/or moving images are read in in order to simulate a course of patient kinematics and to determine, by inverse kinematics, a time course of the predefined forces and momenta on and at the implant model, which are used as a basis for simulated dynamic loads in the step of calculating simulated dynamic loads.

24. The computer-implemented implant assistance method according to claim 13, further comprising the steps of:
creating a multidimensional matrix based on a variance of input parameters; and
determining limits/boundary parameters that are to be complied with in a combination of input parameters so that the load limits are complied with.

25. The computer-implemented implant assistance method according to claim 24, further comprising the step of outputting, as a function of a patient's weight and an implant size, of an output parameter varus/valgus angle at which the implant model is insertable without exceeding the load limits.

26. The computer-implemented implant assistance method according to claim 24, further comprising the step of outputting a confirmation when a combination of the input parameters is within the limits/boundary parameters.

27. The computer-implemented implant assistance method according to claim 24, further comprising the step of outputting a warning when a combination of the input parameters is outside the limits/boundary parameters.

28. The computer-implemented implant assistance method according to claim 13, wherein the at least one implant parameter is at least one implant dimension and/or a weight restriction of a selected implant model.

29. The computer-implemented implant assistance method according to claim 13, wherein the dynamic activity is walking or running.

30. A computer-readable storage medium comprising instructions which, when executed by a computer, cause the computer to perform the computer-implemented implant assistance method according to claim 13.

31. An implant assistance system for a simulation and selection of at least one implant for optimized insertion or joint replacement in a surgical intervention in a patient, the implant assistance system comprising:
A. a display device for outputting a visual content; and
B. a control unit configured for:
i. reading in at least one implant parameter of an implant model wherein the at least one implant parameter comprises an implant type;
ii. calculating simulated dynamic loads based on the at least one implant parameter by predefined forces and momenta on and at the implant model of a load situation of a dynamic activity;
iii. determining, based on the at least one implant parameter, whether load limits of the implant model are complied with in the simulated dynamic loads; and
iv. outputting, by the display device:
a visual confirmation of a selection of the implant model when the load limits are complied with, or
a visual warning before the selection of the implant model when the load limits are exceeded.

32. The implant assistance system according to claim 31, wherein the at least one implant parameter is at least one implant dimension and/or a weight restriction of the implant model.
